(19) United States
(12) Patent Application Publication
(10) Pub. No.: US 2026/0004607 A1
(43) Pub. Date: Jan. 1, 2026

(54) DEVICE FOR CONTACTLESS ACQUISITION OF BIOMETRIC DATA FROM SKIN AREAS

(71) Applicant: IDLOOP GMBH, Jena (DE)

(72) Inventors: Tom MICHALSKY, Zwenkau (DE); Daniel GLÄSNER, Jena (DE); Jorg REINHOLD, Jena (DE); Philipp RIEHL, Jena (DE)

(21) Appl. No.: 18/878,295

(22) PCT Filed: Jun. 19, 2023
(86) PCT No.: PCT/EP2023/066503

§ 371 Date: Dec. 23, 2024

(30) Foreign Application Priority Data
Jun. 24, 2022 (DE) 10 2022 115 810.1

(51) Int. Cl. G06V 40/13; A61B 5/1172; G06T 17/00; G06V 10/145; G06V 10/147

(52) U.S. Cl. CPC G06V 40/1312; A61B 5/1172; G06T 17/00; G06V 10/145; G06V 10/147; G06V 40/1318; G06V 40/1324; G06T 2210/56

(57) ABSTRACT

The present disclosure relates to a device 1000 for contactless acquisition of biometric data from skin areas. For example, a sensor optics arrangement 120 of the image acquisition unit 100 or an emitter optics arrangement 220 of the illumination unit 200 is offset such that the focal planes 135, 235 of the image acquisition unit 100 and the illumination unit 200 essentially completely overlap. As a result, for example, the area of the optical sensor 115 of the image acquisition unit 100 or the area of the light emitter 215 of the illumination unit 200 can be used to the maximum, thereby resulting in a significantly improved quality of the acquisition of the biometric features of a hand, in particular the fingers. In addition, the compactness of the device 1000 can be further increased, for example by beam folding using reflective optical elements 700.

### DESCRIPTION

The present disclosure relates to a device for contactless acquisition of biometric data from skin areas.

### BACKGROUND

In the wake of pandemics and rapidly spreading infections, it is becoming increasingly important to acquire biometric data from people in a contactless manner, for example, in the case of fingerprints, without touching surfaces.

Several so-called fingerprint scanners are already known from the prior art, for example from US 2009/046 331 A1 or from US 2012/076 369 A1, which are set up for contactless acquisition of fingerprints, for example by using a camera as an image acquisition unit and one or more light sources or projectors as an illumination unit.

One problem that such systems often have is the combination of the requirement for such systems to be compact while maintaining high quality of the fingerprints acquired, especially if these systems are to be used for governmental purposes, for example, as the requirements here are noticeably higher than in the private or business sector, for example.

To make the systems compact, attempts are often made to position the camera and the projector as close to each other as possible within the device and to resolve the overlap of the depth of field of the camera with the depth of field of the projector by using a Scheimpflug arrangement of camera and projector. However, this structure leads to a distortion of the image and thus to a partial deterioration in the quality of the acquired fingerprints within the image.

In view of the disadvantages described above, and based on the prior art described above, it is an object of the present application to be able to provide an improved device for the contactless acquisition of biometric data from skin areas with improved quality of the acquired fingerprints while at the same time maintaining a compact structure.

### SUMMARY

The present disclosure relates to a device for contactless acquisition of biometric data from skin areas.

In particular, to achieve the above-mentioned object, a device for contactless acquisition of biometric data from skin areas according to claim 1 is proposed. The dependent claims relate to some exemplary preferred embodiments.

According to one aspect, in some embodiments, a device for contactless acquisition of biometric data from skin areas is proposed, comprising: at least one image acquisition unit, which comprises an optical sensor and a sensor optics arrangement arranged upstream in the beam path and is configured for contactless acquisition of image data from illuminated skin areas, and at least one illumination unit, which comprises a light emitter and an emitter optics arrangement arranged downstream in the beam path and is configured to illuminate the skin areas to be acquired by the image acquisition unit, wherein the sensor optics arrangement is arranged offset relative to the optical sensor along the optical main planes of the sensor optics arrangement or the emitter optics arrangement is arranged offset relative to the light emitter along the optical main planes of the emitter optics arrangement, so that the focal plane section of the image acquisition unit and the focal plane section of the illumination unit have the greatest possible overlap with one another.

It was advantageously recognized that by providing an offset of a sensor optics arrangement and/or an offset of an emitter optics arrangement in a device for contactless acquisition of biometric data, the quantity as well as the quality of the acquired biometric features can be increased, since both the focal plane of the image acquisition unit can be significantly better superimposed/overlapped with the focal plane of the illumination unit, and no distortion is caused when acquiring the biometric features by the image acquisition unit, since a Scheimpflug arrangement can be avoided.

It was also advantageously recognized that when using structured light to illuminate the skin areas, 3D data of the skin areas, in particular the biometric features of the hand or fingers such as the papillaries/papillary ridges or the valleys of the valley structure of the skin areas, can be generated in an advantageous manner.

In addition, it was advantageously recognized that an offset of the sensor optics arrangement and/or an offset of the emitter optics arrangement can lead to reflective optics elements being provided within the device, since the image acquisition unit and the illumination unit can be moved further apart from each other.

In addition, it was advantageously recognized that the reflective optical elements, which can fold the beam path of the image acquisition unit and/or the illumination unit, allow the device to be designed more compactly and still maintain the working distance that is advantageous for acquiring the biometric features using the image acquisition unit and illumination unit, even if this is longer than the external dimensions of the device itself.

In sum, the recognized advantages can lead to a more compact device with significantly improved image quality or data quality of the acquired biometric data being provided for the contactless acquisition of biometric features.

In some preferred embodiments, the offset of the sensor optics arrangement relative to the optical sensor or the offset of the emitter optics arrangement relative to the light emitter can be selected such that the overlap of the focal plane sections of the image acquisition unit and illumination unit relative to the maximum possible overlap of the focal plane sections of the image acquisition unit and illumination unit is at least 80%, in particular greater than 95%.

In some preferred embodiments, the offset of the sensor optics arrangement relative to the optical sensor can have an offset between <150% and >50%, in particular an offset between <110% and >90%, or the offset of the emitter optics arrangement relative to the light emitter can have an offset between <60% and >40%.

In some preferred embodiments, the sensor optics arrangement can be arranged offset relative to the optical sensor along the main planes of the sensor optics arrangement and the emitter optics arrangement can be arranged offset relative to the light emitter along the main planes of the emitter optics arrangement.

In some preferred embodiments, the offset of the sensor optics arrangement relative to the optical sensor and the offset of the emitter optics arrangement relative to the light emitter can each have an offset >25%.

In some preferred embodiments, the device can further have a system area in which the units of the device are provided and an acquisition area for the contactless acquisition of image data of the skin areas to be acquired, wherein the system area and the acquisition area are separated from one another by a common interface.

In some preferred embodiments, the focal plane section of the image acquisition unit and/or the illumination unit can be arranged in the acquisition area, wherein the beam path of the image acquisition unit and/or the beam path of the illumination unit is folded for contactless acquisition of the skin areas in the focal plane section by means of reflective optical elements, so that the working distance of the sensor optical arrangement and/or the emitter optical arrangement to the respective focal plane section is greater than one of the external dimensions in depth, width and height of the system area of the device.

In some preferred embodiments, the reflective optical elements can comprise mirrors and/or prisms.

In some preferred embodiments, the acquisition area having the focal plane sections of the image acquisition unit and the illumination unit can be formed such that the skin areas to be acquired can be positioned within the depth of field of the focal plane sections of the image acquisition unit and the illumination unit.

In some preferred embodiments, the skin areas to be acquired can include areas of the human hand, in particular the palm and the fingers, and the acquisition area can be formed such that several fingers can be acquired simultaneously by the image acquisition unit.

In some preferred embodiments, the acquisition area can be formed such that 4 fingers, in particular the index, middle, ring and little fingers, or 2 thumbs, or the entire palm, or the entire inside of the hand can be acquired simultaneously by the image acquisition unit.

In some preferred embodiments, the illumination unit can illuminate the skin areas with structured light.

In some preferred embodiments, the device can also be configured to generate 3D data of the skin areas, in particular as a 3D point cloud, based on the image data of the skin areas acquired by the at least one image acquisition unit that were illuminated using structured light.

In some preferred embodiments, the light emitter can emit light at a wavelength between 400 nm and 550 nm, particularly preferably between 450 nm and 500 nm.

In some preferred embodiments, the system area of the device can have external dimensions in depth, width and height of at most 8″, preferably <180 mm, particularly preferably <160 mm.

In some preferred embodiments, the device including the system area and acquisition area can have external dimensions in depth, width and height of at most 8″, preferably <180 mm, particularly preferably <160 mm.

Further aspects and their advantages as well as advantages and more specific implementation options of the aspects and features described above are described in the following descriptions and explanations of the attached figures, which are not to be construed as limiting in any way.

### BRIEF DESCRIPTION OF THE FIGURES

FIG. 1 shows an example arrangement of an image acquisition unit and an illumination unit, the depth of field/focal planes of which can overlap by way of example,

FIG. 2 shows an example of an image acquisition unit and an illumination unit with crossed focal planes (see illustration (a)) and in a Scheimpflug arrangement (see illustration (b)), whereby the depth of field/focal planes can overlap by way of example,

FIG. 3 shows an embodiment of a device for contactless acquisition of biometric data from skin areas, in which, for example, the sensor optics arrangement is shifted/offset relative to the camera and the emitter optics arrangement is shifted/offset relative to the projector along their respective optical main planes,

FIG. 4 shows, for example, the offset of the illuminated section of the focal plane of the illumination unit caused by the offset of the emitter optics arrangement of the projector,

FIG. 5 shows, by way of example, the difference between an image acquired by means of the Scheimpflug arrangement (see illustration (b)) and an image acquired by means of an offset of the sensor optics arrangement of the image acquisition unit (see illustration (c)) in comparison to an image acquired by means of a sensor optics arrangement and/or emitter optics arrangement arranged symmetrically with respect to the optical sensor and/or light emitter (see illustration (a)),

FIG. 6 shows, by way of example, an embodiment of a device with an image acquisition unit, illumination unit and computing unit, wherein the beam paths are folded by means of reflective optics/reflective optical elements,

FIG. 7 shows, by way of example, a further advantage of the offset of the sensor optics arrangement with respect to the camera and/or the emitter optics arrangement with respect to the projector in relation to the required space,

FIG. 8 shows, by way of example, a further advantage of the offset of the sensor optics arrangement with respect to the camera and/or the emitter optics arrangement with respect to the projector in relation to the ease of use.

### DETAILED DESCRIPTION OF THE FIGURES AND PREFERRED EMBODIMENTS

In the following, examples or embodiments of the present disclosure are described in detail with reference to the attached figures. Identical or similar elements in the figures can be designated with the same reference signs, but sometimes also with different reference signs.

It should be emphasized that the subject matter of the present disclosure is in no way limited or restricted to the embodiments described below and their features, but also includes modifications of the embodiments, in particular those that are covered by modifications of the features of the examples described or by combining one or more of the features of the examples described within the scope of protection of the independent claims.

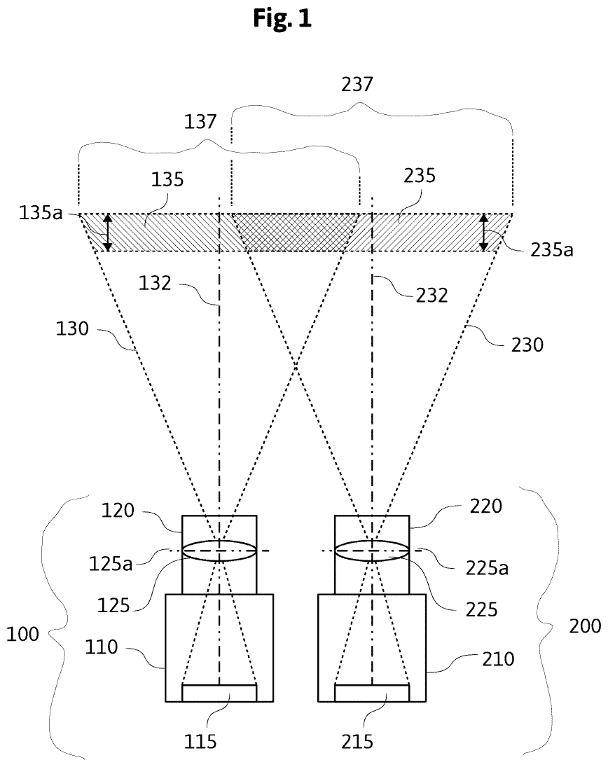

FIG. 1 shows an example arrangement of an image acquisition unit 100 and an illumination unit 200, the depth of field/focal planes 135, 235 of which can overlap by way of example.

This shows, by way of example, how the beam path 130 of the image acquisition unit 100 applied by the camera 110 and the sensor optics arrangement 120 (for example as the lens of the camera 110) can overlap with the beam path 230 formed by the projector 210 and the emitter optics arrangement 220 (for example as the lens of the projector 210) in the region of the respective focal planes 135 and 235 (see the cross-hatched area).

The beam path 130 (with the optical axis 132) of the image acquisition unit 100 can be applied by means of an optical system, for example comprising the optical sensor 115 of the camera 110 and the lens/lens system 125 (with corresponding optical main planes 125a) of the sensor optics arrangement 120.

Something similar can apply to the beam path 230 (with the optical axis 232) of the illumination unit 200, wherein the beam path 230 can be formed by means of an optical system, for example comprising the light emitter 215 of the projector 210 and the lens/lens system 225 (with corresponding optical main planes 225a) of the emitter optics arrangement 220.

For example, the sharpness plane/focus plane 135 of the image recording unit 100 and the depth of field/focal plane of the illumination unit 235 can be limited to a respective section 137, 237 due to the respective beam path 130, 230, which can each be influenced essentially by the optical sensor 115 and the sensor optics arrangement 120 and/or by the light emitter 215 and the emitter optics arrangement 220, and focal planes 135, 235 limited to these sections 137, 237 can now, for example, superimpose or overlap in certain areas. In addition, each focal plane 135, 235 has a corresponding depth of field 135a, 235a (see arrows in the obliquely hatched areas of the focal planes 135, 235), within which, for example, objects can be captured essentially sharply by the camera 110 or the structure projected by the projector 210 (for example in the case of structured light) can be sharply imaged on the object.

In the representation of an exemplary interaction between camera 110 and projector 210 shown in FIG. 1, it can be seen that some of the focal planes 135, 235 overlap (shown with cross-hatching), but also a considerable part of these focal planes 135, 235 do not overlap and thus represent dead areas for possible acquisitions/captures of fingerprints of a hand, since in these dead areas either only the projector 210 illuminates or only the camera 110 acquires images.

This can, for example, lead to certain areas of the optical sensor 115 of the camera 110 not being able to be used for acquiring the fingerprints. At the same time, it can lead to the area used by the optical sensor 115 having to have a correspondingly high resolution (correspondingly large number of pixels of the optical sensor 115) in order to ensure the highest possible quality (with a correspondingly high level of detail) of the recorded fingerprints (in particular, for example, for governmental purposes), since only a comparatively small part of the total area of the optical sensor 115 can be used to acquire the biometric features.

In addition, for example, the area of the light emitter 215 of the projector 210 cannot be fully used, so that the potential for simultaneously acquiring the largest possible area of the hand or fingers for the acquisition of biometric data cannot be used due to the limited illumination area available, and this can cause a deficit in the efficiency of the acquisition of the biometric data.

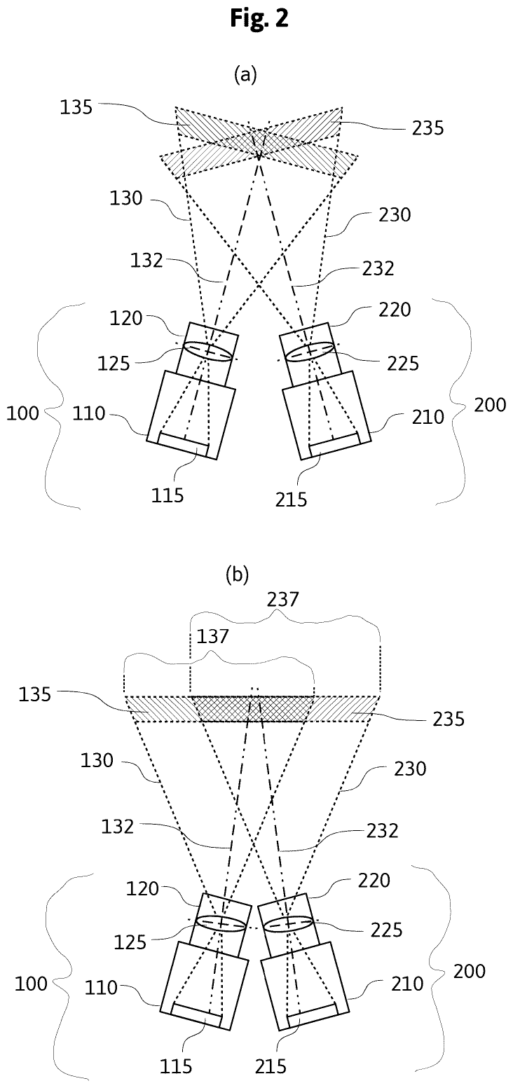

Based on this example of the problem with the superposition of the focal planes 135, 235, contactless fingerprint acquisition systems (fingerprint scanners) can, for example, have a Scheimpflug arrangement of image acquisition unit 100 and illumination unit 200, which is shown and described below in FIG. 2.

FIG. 2 shows an example of an image acquisition unit 100 and an illumination unit 200 with crossed focal planes 135, 235 (see illustration (a)) and in a Scheimpflug arrangement (see illustration (b)), whereby the depth of field planes/focal planes 135, 235 can overlap, for example.

In contrast to the example arrangement of image acquisition unit 100 and illumination unit 200 as shown in FIG. 1, the Scheimpflug arrangement (see illustration (b) of FIG. 2) can now be used to try to maximize the area of overlap of the (sections 137, 237 of the) focal planes 135, 235.

The image acquisition unit 100 and the illumination unit 200 are initially aligned convergently to the measuring area/measurement volume. However, this leads to the fact that the focal planes 135, 235 initially cross/intersect at a certain angle (see illustration (a) of FIG. 2), so that the possible measuring area for acquiring fingerprints is initially even smaller (see the cross-hatched area here too).

To counteract this, the sensor optics arrangement 120 and the emitter optics arrangement 220 are now tilted according to “Scheimpflug”, thereby creating coplanarity in the two focal planes 135, 235 (see illustration (b) of FIG. 2). This can, for example, increase the superposition/overlap of the sections 137, 237 of the focal planes 135, 235 with one another compared to the exemplary arrangement according to FIG. 1.

In addition, the Scheimpflug arrangement could potentially make better use of the areas of the optical sensor 115 and light emitter 215.

One major problem that the Scheimpflug arrangement can cause, however, is a perspective distortion of the image of the depth of field plane in the image acquisition unit 100; converging lines can occur in the acquired/captured image (see illustration (b) of FIG. 5). The measuring area in the camera can be displayed geometrically distorted, whereby an attempt can be made to compensate for this disadvantage, for example by using a higher resolution of the optical sensor 115 (higher number of pixels of the optical sensor 115), in order to achieve a minimum resolution specified for the respective application in all image areas (for example, private or business areas or, with significantly higher requirements, in the governmental area).

However, the illumination by the illumination unit 200 can also experience negative influences for the same reasons, so that, for example, the measuring area/the measuring volume is illuminated unevenly, for example, the measuring object is illuminated too much from the side (shadow formation at certain points on the object surface to be acquired) and, for example in the case of structured light projected onto the finger/hand, a distortion of this projected structure.

In order to counteract this problem and thereby advantageously maximize both the overlap of the sections 137, 237 of the focal planes 135, 235 and the use of the area of the optical sensor 115 of the image acquisition unit 100 and the use of the area of the light emitter 215 of the illumination unit 200, the device 1000 for the contactless acquisition of biometric data from skin areas is to be provided as an example and is explained below.

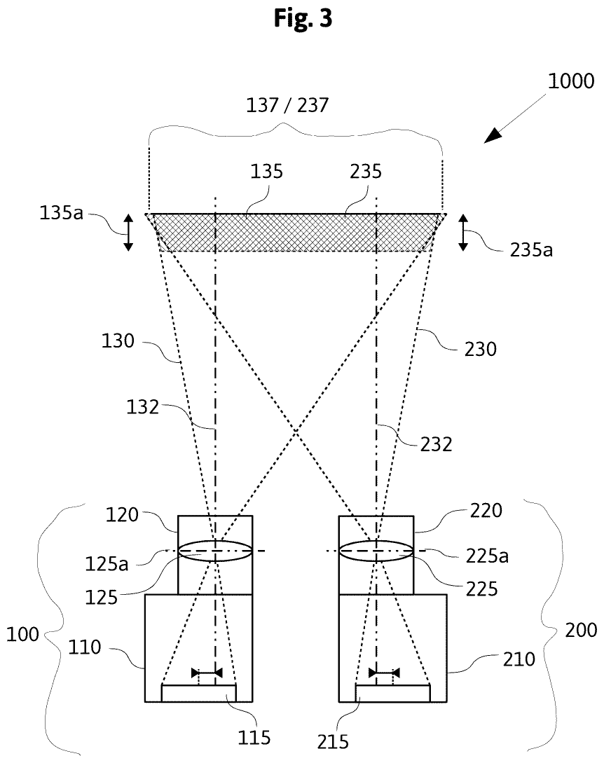

FIG. 3 shows an embodiment of a device 1000 for contactless acquisition of biometric data from skin areas, in which, for example, the sensor optics arrangement 120 is shifted/offset relative to the camera 110 and the emitter optics arrangement 220 is shifted/offset relative to the projector 210 along their respective optical main planes 125a, 225a.

The device 1000 shown here by way of example comprises, as described in FIG. 1, an image acquisition unit 100 comprising a camera 110 with an optical sensor 115 (for example a CMOS sensor, but can also comprise an optical sensor of a different type) and a sensor optics arrangement 120 with a lens/lens system 125 having the optical main plane 125a, as well as an illumination unit 200 comprising a projector 210 with a light emitter 215 (for example for emitting structured light) and an emitter optics arrangement 220 with a lens/lens system 225 having the optical main plane 225a.

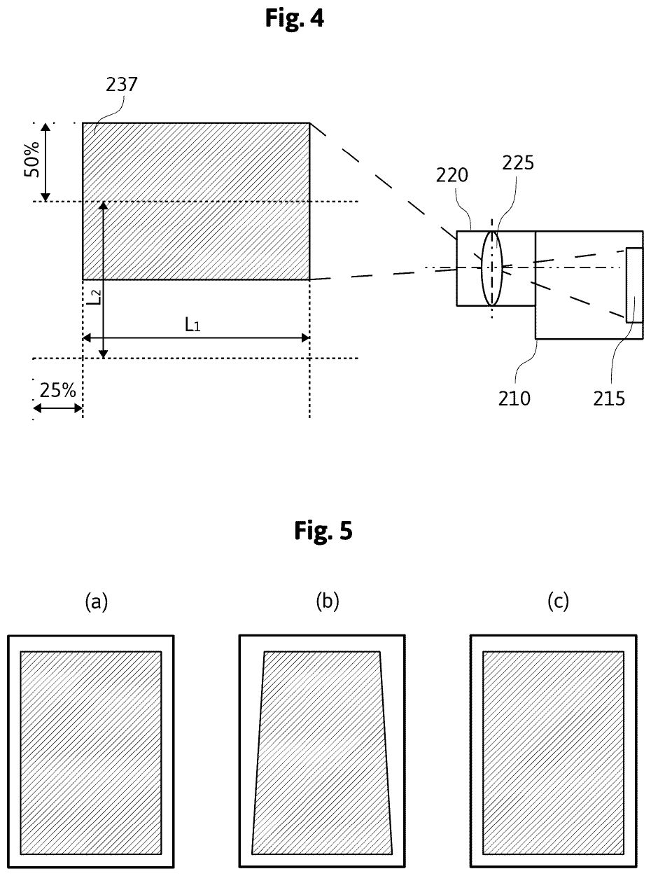

For example, the sensor optics arrangement 120 (measured at its optical axis 132) can be arranged offset from the line of symmetry that is perpendicular to the optically effective surface of the optical sensor 115 of the camera 110 and is arranged essentially in the middle of the optically effective surface of the optical sensor 115, and thereby have an offset (for explanations of the offset, see FIG. 4 and the associated description). This can lead to the beam path 130 usable by the optical sensor 115 of the camera 110, and in particular the section 137 of the focal plane 135 of the image acquisition unit 100 being offset/shifted (shifted/offset along their respective optical main plane 125a) towards the section 237 of the focal plane 235 of the illumination unit 200.

Conversely, alternatively or in addition to the offset of the sensor optics arrangement 120 of the image acquisition unit 100, for example, the emitter optics arrangement 220 (measured at its optical axis 232) can be arranged offset from the line of symmetry that is perpendicular to the optically effective surface of the light emitter 215 of the projector 210 and arranged essentially in the middle of the optically effective surface of the light emitter 215 and thus also have an offset (for explanations of the offset, see FIG. 4 and the associated description). This can, comparable to the image acquisition unit 100, lead to the beam path 230 (of the projected light) usable by the light emitter 215 of the projector 210, and in particular the section 237 of the focal plane 235 of the illumination unit 200, being offset/shifted (shifted/offset along their respective optical main planes 225a) towards the section 137 of the focal plane 135 of the image acquisition unit 100.

Advantageously, the embodiment explained by way of example makes it possible to dispense with any tilting of the lens/lens system 125, 225 of the sensor optics arrangement 120 or the emitter optics arrangement 220 (as in the Scheimpflug arrangement described in illustration (b) in FIG. 2), and yet the overlap of the two sections 137, 237 of the two focal planes 135, 235 (focus planes 135, 235), which are also aligned essentially parallel to one another, can be maximized, sometimes even up to an overlap of more than 95% (where 100% overlap means that at least one section 137, 237 of the focal planes 135, 235 is completely overlaid/overlapped by the other section 137, 237 of the focal planes 135, 235).

In particular, the overlap of the two sections 137, 237 of the focal planes 135, 235 can be at least 80%, preferably at least 85% and particularly preferably at least 90% of the maximum possible overlap, so that the maximum usable area of the optical sensor 115 of the camera 110 and/or the maximum usable area of the light emitter 215 of the projector 210 can be used as much as possible in order to capture/acquire the biometric features such as fingerprints and/or the features of an entire palm with the largest possible illumination area (due to the improved use of the optically effective surface of the light emitter 215) and the highest possible resolution (number of pixels) of the optical sensor 115 within the largest possible illumination area.

Due to the offset of the sensor optics arrangement 120 and the emitter optics arrangement 220 described as an example, the focal planes 135, 235 can be aligned essentially congruently with one another and thereby maintain their essentially parallelism to one another, which in particular enables a sharp 3D acquisition of the biometric features such as fingerprints etc. with a high level of detail in all areas of the sections 137, 237 of the focal planes 135, 235, for example as a 3D point cloud.

The embodiment described as an example with the offset of the sensor optics arrangement 120 and the emitter optics arrangement 220 can offer an optimal overlap of the section 237 as the illumination zone and the section 137 as the observation zone of the measuring area/measurement volume. The optical sensor 115 of the camera 110 and also the light emitter 215 of the projector 210 therefore do not require more pixels (resolution) than absolutely necessary, since in the best case scenario no dead zones arise when the sections 137, 237 overlap.

For an exemplary configuration of the illumination unit 200, for example, the light emitter 215 of the projector 210 can emit light at a wavelength between 400 nm and 550 nm, particularly preferably between 450 nm and 500 nm, and project it onto the skin areas to be acquired (such as fingers or palms).

In addition, the light projected by the projector 210 can be formed as structured light, so that a predetermined pattern/structure is projected onto the biometric features to be acquired, for example in order to make the biometric features of the hand or fingers such as the papillaries/papillary ridges or the valleys of the valley structure of the skin areas more efficiently recognizable for the image acquisition unit 100 and to make it easier to generate 3D data (for example as a 3D point cloud) from them.

For an exemplary configuration of the image acquisition unit 100, the optical sensor 115 can, for example, be formed as a CMOS sensor (or APS) or as a CCD sensor and have a resolution of 0.3 megapixels (for example for very small objects to be recorded such as a fingertip) to 50 megapixels (for example for very large objects such as the entire hand).

It should be noted at this point that the lens 125, 225 or the lens system 125, 225 shown in FIG. 3 is only an example and should not be understood as a restrictive design. The lens 125, 225/the lens system 125, 225 can be a wide variety of types of lenses, for example converging or scattering lenses or a combination thereof, whereby, for example, an aperture may be included or no aperture may be included, as well as certain filters or no filters may be included.

It should also be noted at this point that the beam paths 132, 232 shown in the figures are only examples and should not be understood as restrictive. Depending on the specific configuration of the sensor optics arrangement 120 or the emitter optics arrangement 220, the beam paths 132, 232 can have different courses.

FIG. 4 shows, by way of example, the offset of the illuminated section 237 of the focal plane 235 of the illumination unit 200 caused by the offset of the emitter optics arrangement 220 of the projector 210.

The offset is defined in percent as the relative shift of the illumination area (section 237) generated by the illumination unit 200 in respect to the area size, which in the present example is shown by the dimensions L1 and L2.

If, for example, an offset of ±50% is made along the dimension L2 of the originally projected illumination area, as shown by way of example in FIG. 4, the section 237 is offset by half (±50%) of its dimension L2 along its dimension L2.

Of course, an offset can also be made along the dimension L1 (as shown by way of example with the ±25% of the dimension L1 along the dimension L1) or a superimposed offset of both dimensions L1 and L2.

The example shown here relates to the offset of the illumination unit 200, whereby this offset can also be applied analogously to the image acquisition unit 100.

In addition, the offset can also be defined as the ratio of the lateral displacement of the respective optics arrangement (sensor optics arrangement 120 or emitter optics arrangement 220) to the respective chip length of the optical sensor 115 or light emitter 215 in the direction of displacement (for example along the optical main planes 125a of the sensor optics arrangement 120 or along the optical main planes 225a of the emitter optics arrangement 220).

For example, in the device 1000, the offset of the sensor optics arrangement 120 relative to the optical sensor 115 can have an offset between <150% and >50%, preferably <130% and >70%, particularly preferably <110% and >90%.

Alternatively or additionally, for example, in the device 1000, the offset of the emitter optics arrangement 220 relative to the light emitter 215 can have an offset between <60% and >40%, preferably approximately 50%.

In addition, for example, the offset of the sensor optics arrangement 120 relative to the optical sensor 115 and the offset of the emitter optics arrangement 220 relative to the light emitter 215 can each have an offset of >25%.

All of these exemplary embodiments of the offset of the sensor optics arrangement 120 relative to the optical sensor 115 or of the emitter optics arrangement 220 relative to the light emitter 215 can lead to an optimized or optimal overlap of the illumination area (section 237) of the illumination unit 200 with the measurement area (section 137) that can be captured by the image acquisition unit 100 within their respective depth of field 135a, 235a.

FIG. 5 shows, by way of example, the difference between an image acquired by means of the Scheimpflug arrangement and an image acquired by means of an offset of the sensor optics arrangement 120 of the image acquisition unit 100 in comparison to an image acquired by means of a sensor optics arrangement 120 and/or emitter optics arrangement 220 arranged symmetrically with respect to the optical sensor 115 and/or light emitter 215.

In the illustration (a) of FIG. 5, a hatched area can be seen as an example image of how it would be acquired by the camera if there were neither a Scheimpflug arrangement nor an offset of the sensor optics arrangement 120 and/or emitter optics arrangement 220, but instead the sensor optics arrangement 120 and/or emitter optics arrangement 220 were arranged symmetrically with respect to the optical sensor 115 and/or the light emitter 215. As can be seen by way of example, the hatched area in illustration (a) is a rectangle with vertical side lines and horizontal upper and lower edges.

In comparison, in illustration (b) of FIG. 5, which now reproduces the image using a camera 110 in Scheimpflug arrangement (see also FIG. 2, illustration (b)), it is very clearly visible how the original, hatched rectangle is distorted and becomes more of a type of trapezoid with an upper, narrower (and thus distorted) part of the image and a lower part that is of normal width compared to the original image.

As already described, in order to meet a required minimum quality (minimum resolution) of the objects captured in the images, for example, it may be necessary to use a much higher resolution optical sensor 115 to reproduce the distorted areas of the image (and thus distorted objects captured in the image) in the required resolution, whereas in the undistorted areas a simpler (lower resolution) optical sensor 115 would have been sufficient. This can also lead to the costs of a device being correspondingly higher due to the selected Scheimpflug arrangement.

In further comparison, one can see in illustration (c) of FIG. 5 how the image captured by means of an offset sensor optics arrangement 120 is essentially the same as the original image. There is no distortion here from the image acquisition unit 100. Likewise, there would be no distortion in a projected structure or shadowing effects or uneven illumination from the illumination unit 200.

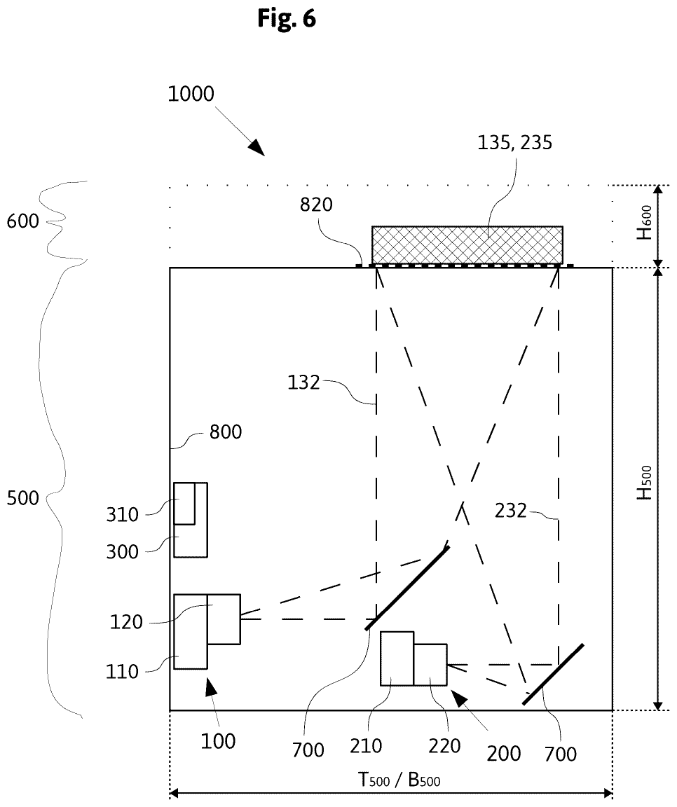

FIG. 6 shows an example of an embodiment of a device 1000 with image acquisition unit 100, illumination unit 200 and computing unit 300, wherein the beam paths 132, 232 are folded by means of reflective optics 700/reflective optical elements 700.

The device 1000 shown here as an example is divided into a system area 500 and an acquisition area 600, which can be separated from one another by a common interface, for example an optically transparent element 820 (such as a glass plate 820). However, the interface can also be part of a housing 800, or a combination of housing 800 and the optically transparent element 820, or separate the acquisition area 600 from the system area 500 without any physical form.

In addition, the device 1000 shown as an example in FIG. 6 can have a camera 110 with a sensor optics arrangement 120 and a projector 210 with an emitter optics arrangement 220, wherein both the sensor optics arrangement 120 is arranged offset from the camera 110 and the emitter optics arrangement 220 is arranged offset from the projector 210, as described in FIG. 3, so that, for example, both the image acquisition unit 100 and the illumination unit 200 each have an offset. It should already be pointed out at this point that only one of the image acquisition unit 100 and illumination unit 200 can have an offset.

The beam path 132 of the image acquisition unit 100 and the beam path 232 of the illumination unit 200 are each deflected/folded within the housing 800 by reflective optical elements 700, for example, before they reach the common focal plane 135, 235. For example, the beam paths 132, 232 run through an optically transparent element 820, for example in the form of a glass plate 820, before they reach or form the common focal plane 135, 235.

It should be mentioned at this point that the optically transparent element 820 does not have to be present, but can also be omitted or replaced by another element. The optically transparent element 820/the glass plate 820 can advantageously be used to protect, for example, the image acquisition unit 100, illumination unit 200 and/or computing unit 300 located in the system area 500, for example, from contamination, damage and/or manipulation.

The reflective optical elements 700 can be formed, for example, as mirrors and/or beam-deflecting prisms, or as a combination of these elements (mirror/prism).

As shown by way of example, the image acquisition unit 100, the illumination unit 200, the computing unit 300 and the reflective optical elements 700 are provided/arranged within the system area 500 of the device 1000. The common focal plane 135, 235 of the image acquisition unit 100 and the illumination unit 200 are located, for example, outside the system area 500 and within the acquisition area 600.

The two areas (acquisition area 600 and system area 500) can, for example, have a comparatively compact design, since the reflective optical elements 700 fold/deflect the beam paths 132, 232 in such a way that the desired working distance (distance between the sensor optics arrangement 120 or emitter optics arrangement 220 to the focal plane 135, 235 along the respective beam path 132, 232) corresponds to, for example, approximately twice the diameter (or diagonal) of the sections 137, 237 of the focal planes 135, 235 (for example, a working distance of approximately 200 mm with approximately 100 mm diameter/diagonal of the sections 137, 237), although the external dimensions in depth T500, width B500 and height H500 of the system area 500 are, for example, each at most 8″, preferably less than 180 mm, and particularly preferably less than 160 mm.

A sufficiently large working distance can be particularly advantageous in order to avoid any shadowing effects on the biometric features (such as fingerprints or the valleys of the valley structures of the papillaries) during the acquisition of the biometric features. It can therefore be advantageous, for example, that the beam path(s) 132, 232 are not guided directly from the camera 110 or the projector 210 to the object to be captured (for example the palm of the hand or the fingers).

The folding/deflection of the beam paths 132, 232 by means of the reflective optical elements 700 can be advantageously supported or enabled, for example, by the offset of the sensor optics arrangement 120 relative to the camera 110 and/or by the offset of the emitter optics arrangement 220 relative to the projector 210.

It can be advantageous that by offsetting the sensor optics arrangement 120 relative to the camera 110 and/or by offsetting the emitter optics arrangement 220 relative to the projector 210, at least one reflective optic 700 can be positioned in the respective beam path 132, 232 without covering the other beam path 132, 232 (or even only partially), since, with the same overlap in the focal plane 135, 235, the camera 110 and the projector 210 can be moved further apart.

In addition, it can be advantageous to fold the beam paths 132, 232 not just once, as shown in FIG. 6, but to fold them several times if necessary, for example two or three times, in order to be able to make the device 1000 even more compact, for example.

In addition, the system area 500 can be further reduced in size, for example depending on the possible arrangement of the reflective optical elements 700 and/or depending on the number of reflective optical elements 700, so that, for example, the external dimensions in depth T500, width B500 and height H500+H600 of the system area 500 including the acquisition area 600 are each, for example, at most 8″, preferably less than 180 mm, and particularly preferably less than 160 mm.

Furthermore, it can be advantageous if the acquisition area 600 having the sections 137, 237 of the focal planes 135, 235 of the image acquisition unit 100 and the illumination unit 200 is formed such that the skin areas to be acquired can be positioned within the depth of field 135a, 235a of the sections 137, 237 of the focal planes 135, 235 of the image acquisition unit 100 and the illumination unit 200.

This makes it possible, for example, to effectively acquire the biometric features of the skin areas (for example fingerprints and/or palms) in high quality (with a high level of detail), so that high-quality 3D data can later be created from them for the respective acquired biometric features.

In particular, it can be advantageous if the skin areas to be acquired include areas of the human hand, in particular the palm and the fingers, and the acquisition area 600 is formed such that several fingers can be acquired simultaneously by the image acquisition unit 100. This can further increase the efficiency of the acquisition of the biometric features.

For example, the acquisition area 600 can be formed such that 4 fingers, in particular the index, middle, ring and little fingers, or 2 thumbs, or the entire palm, or the entire inside of the hand can be acquired simultaneously by the image acquisition unit 100.

In addition, for example, the system area 500 can have a computing unit 300 for controlling the image acquisition unit 100 and illumination unit 200 and for processing the acquired biometric data, whereby the computing unit 300 can alternatively also be provided outside the system area 500. For example, the computing unit 300 of the device 1000 can process the acquired biometric data (for example, convert the image data acquired by the image acquisition unit 100 into biometric data of the skin areas, for example into 3D data, in particular into a 3D point cloud) and optionally store it in a storage unit 310, whereby the data can also be stored outside the device 1000.

In addition, the computing unit 300 can compare the processed/calculated biometric data with biometric data already stored in the storage unit 310 and thus detect potential matches.

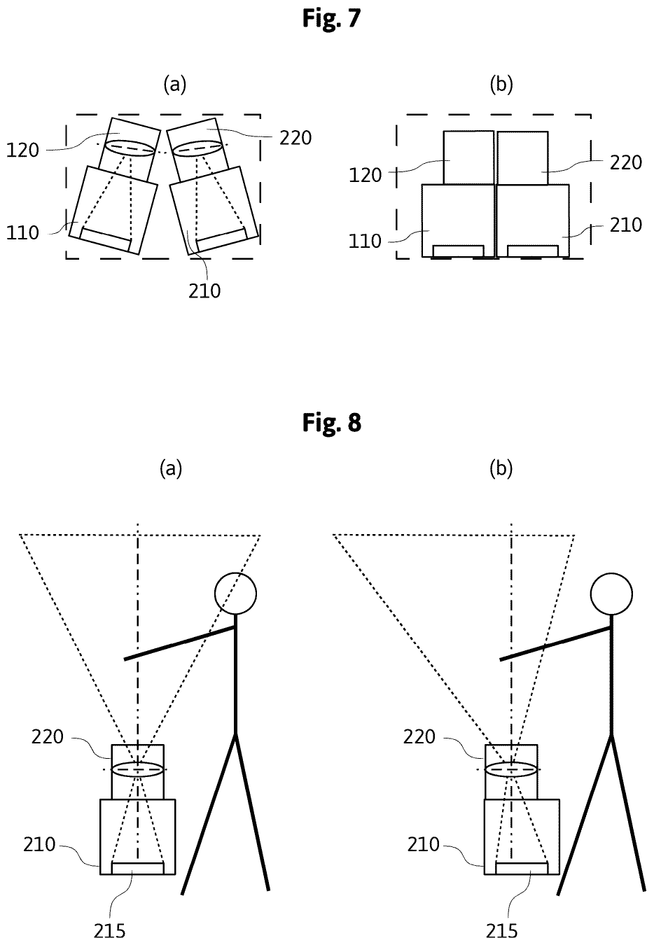

FIG. 7 shows, by way of example, a further advantage of the offset of the sensor optics arrangement 120 relative to the camera 110 and/or the emitter optics arrangement 220 relative to the projector 210 in relation to the required space.

The illustration (a) of FIG. 7 shows the already known arrangement according to Scheimpflug, in which both the image acquisition unit 100 (with camera 110 and sensor optics arrangement 120) and the illumination unit 200 (with projector 210 and emitter optics arrangement 220) are tilted convergently towards each other.

This can, however, lead to a required free space (see dashed rectangle around the image acquisition unit 100 and illumination unit 200) which is significantly larger compared to the arrangement with the offset of the sensor optics arrangement 120 relative to the camera 110 and the emitter optics arrangement 220 relative to the projector 210, as shown in illustration (b).

Or to put it another way: In addition to the already mentioned advantages of the arrangement with offset of the respective optics arrangements 120, 220 with respect to the camera 110 or projector 210, advantages can also be gained with regard to the required space, so that the device 1000, as shown and described in FIG. 6, for example, can be formed to be more compact than comparable devices that implement the Scheimpflug arrangement.

FIG. 8 shows, by way of example, a further advantage of the offset of the sensor optics arrangement 120 relative to the camera 110 and/or the emitter optics arrangement 220 relative to the projector 210 in terms of user comfort.

In particular, the offset of the emitter optics arrangement 220 relative to the projector 210 can have an advantage over a non-offset emitter optics arrangement 220. In particular, as shown in illustration (a) of FIG. 8, a non-offset emitter optics arrangement 220 can result in the user who wants to have his biometric features acquired/needs to have them acquired has to get so close to the device 1000 (as shown and described in FIG. 6, for example) that he is blinded by the illumination beam projected by the projector 210. This can of course be very unpleasant for the user, especially since a projector can have a very bright and powerful light source (such as the light emitter 215, for example).

On the other hand, the offset of the emitter optics arrangement 220 relative to the projector 210, as shown in illustration (b) of FIG. 8, allows the light beam/light cone emitted by the projector 210 to be directed away from the user, so that the user is not blinded, even if he has to get very close to the device.

It should be noted that only examples or embodiments of the present disclosure and technical advantages have been described in detail above with reference to the attached figures. However, the present disclosure is in no way limited or restricted to the embodiments described above and their features or their described combinations, but also includes modifications of the embodiments, in particular those which are covered by modifications of the features of the described examples or by combination or partial combination of one or more of the features of the described examples within the scope of protection of the independent claims.

### LIST OF REFERENCE SIGNS

100 image acquisition unit
110 camera
115 optical sensor
120 sensor optics arrangement
125 lens/lens system of the sensor optics arrangement
125a optical main plane of the lens/lens system of the sensor optics arrangement
130 beam path of the image acquisition unit
132 optical axis of the beam path of the image acquisition unit
135 plane of focus/focal plane of the image acquisition unit
135a depth of field of the focal plane of the image acquisition unit
137 section of the focal plane of the image acquisition unit
200 illumination unit
210 projector
215 light emitter
220 emitter optics arrangement
225 lens/lens system of the emitter optics arrangement
225a optical main plane of the lens/lens system of the emitter optics arrangement
230 beam path of the illumination unit
232 optical axis of the beam path of the illumination unit
235 plane of focus/focal plane of the illumination unit
235a depth of field of the focal plane of the illumination unit
237 section of the focal plane of the illumination unit
300 computing unit
310 storage unit of the computing unit
500 system area
600 acquisition area
700 reflective optics/optical elements
800 housing
820 interface between system area and acquisition area/optically transparent element/glass plate
1000 device
B500 width of the system area
H500 height of the system area
T500 depth of the system area
H600 height of the recording area

1. Device for contactless acquisition of biometric data from skin areas, comprising:
at least one image acquisition unit, which comprises an optical sensor and a sensor optics arrangement arranged upstream in the beam path and is configured for contactless acquisition of image data from illuminated skin areas, and
at least one illumination unit, which comprises a light emitter and an emitter optics arrangement arranged downstream in the beam path and is configured to illuminate the skin areas to be acquired by the image acquisition unit,
wherein the sensor optics arrangement is arranged offset relative to the optical sensor along the optical main planes of the sensor optics arrangement or the emitter optics arrangement is arranged offset relative to the light emitter along the optical main planes of the emitter optics arrangement, so that the focal plane section of the image acquisition unit and the focal plane section of the illumination unit have the greatest possible overlap with one another.

2. Device according to claim 1, wherein
the offset of the sensor optics arrangement relative to the optical sensor or the offset of the emitter optics arrangement relative to the light emitter is selected such that the overlap of the focal plane sections of the image acquisition unit and illumination unit relative to the maximum possible overlap of the focal plane sections of the image acquisition unit and illumination unit is at least 80%, in particular greater than 95%.

3. Device according to claim 1, wherein the offset of the sensor optics arrangement relative to the optical sensor has an offset between <150% and >50%, in particular an offset between <110% and >90%, or the offset of the emitter optics arrangement relative to the light emitter has an offset between <60% and >40%.

4. Device according to claim 1, wherein
the sensor optics arrangement is arranged offset relative to the optical sensor along the main planes of the sensor optics arrangement and the emitter optics arrangement is arranged offset relative to the light emitter along the main planes of the emitter optics arrangement.

5. Device according to claim 4, wherein the offset of the sensor optics arrangement relative to the optical sensor and the offset of the emitter optics arrangement relative to the light emitter each has an offset >25%.

6. Device according to claim 1, wherein the device further has a system area in which the units of the device are provided and an acquisition area for the contactless acquisition of image data of the skin areas to be acquired, wherein the system area and the acquisition area are separated from one another by a common interface.

7. Device according to claim 6, wherein
the focal plane section of the image acquisition unit and/or the illumination unit is arranged in the acquisition area, wherein the beam path of the image acquisition unit and/or the beam path of the illumination unit is folded for contactless acquisition of the skin areas in the focal plane section by means of reflective optical elements, so that the working distance of the sensor optical arrangement and/or the emitter optical arrangement to the respective focal plane section is greater than one of the external dimensions in depth, width and height of the system area of the device.

8. Device according to claim 7, wherein
the reflective optical elements comprise mirrors and/or prisms.

9. Device according claim 6, wherein
the acquisition area having the focal plane sections of the image acquisition unit and the illumination unit is formed such that the skin areas to be acquired are positioned within the depth of field of the focal plane sections of the image acquisition unit and the illumination unit.

10. Device according to claim 9, wherein
the skin areas to be acquired include areas of the human hand, in particular the palm and the fingers, and the acquisition area is formed such that several fingers are acquired simultaneously by the image acquisition unit.

11. Device according to claim 10, wherein
the acquisition area is formed such that
4 fingers, in particular the index, middle, ring and little fingers,
2 thumbs,
the entire palm, or
the entire inside of the hand
is acquired simultaneously by the image acquisition unit.

12. Device according to claim 1, wherein
the illumination unit illuminates the skin areas with structured light.

13. Device according to claim 12, wherein
the device is configured to generate 3D data of the skin areas, in particular as a 3D point cloud, based on the image data of the skin areas acquired by the at least one image acquisition unit that were illuminated using structured light.

14. Device according to claim 1, wherein
the light emitter emits light at a wavelength between 400 nm and 550 nm, particularly preferably between 450 nm and 500 nm.

15. Device according to claim 6, wherein
the system area of the device has external dimensions in depth, width and height of at most 8″, preferably <180 mm, particularly preferably <160 mm.

16. Device according to claim 6, wherein
the device including the system area and acquisition area has external dimensions in depth, width and height of at most 8″, preferably <180 mm, particularly preferably <160 mm.
